(19) United States
(12) Patent Application Publication
(10) Pub. No.: US 2012/0148529 A1
(43) Pub. Date: Jun. 14, 2012

(54) Use of Inclusion Bodies as Therapeutic Agents

(75) Inventors: Elena Garcia-Fruitos, Bellaterra (ES); Esther Vazquez Gomez, Bellaterra (ES); Jose Luis Corchero, Nieto (ES); Antonio Pedro Villa Verde Corrales, Bellaterra (ES)

(73) Assignees: Centro de Invesigacion Biomedica en Red en Bioiagenieria, Biomateriales, y Nanomedicina (Cibe, Zaragoza (ES); Universitat Autonoma de Barcelona et al., Barcelona (ES)

(21) Appl. No.: 13/319,772

(22) PCT Filed: May 12, 2010
(86) PCT No.: PCT/IB2010/001330

§ 371 Date: Jan. 26, 2012

(30) Foreign Application Priority Data
May 12, 2009 (EP) 09382069.4

(51) Int. Cl. A61K 38/20; A61K 38/21; A61K 38/19; A61K 39/395; A61K 35/12; A61K 38/44; A61K 33/24; C12N 5/00; A61K 38/00; A61P 1/16; A61K 38/18; A61P 7/00; A61K 38/30; A61P 7/06; A61P 25/16; A61P 25/28; A61K 38/24; A61K 38/09; A61K 38/35; A61K 38/11; A61K 38/25; A61K 38/22; A61K 38/37; A61P 35/00; A61P 35/02; A61P 31/14

(52) U.S. Cl. 424/85.2; 424/85.1; 424/85.4; 424/85.5; 424/85.6; 424/85.7; 424/130.1; 424/93.21; 424/93.7; 424/94.4; 424/649; 435/325; 514/1.1; 514/5.9; 514/7.7; 514/8.1; 514/8.2; 514/8.4; 514/8.5; 514/8.8; 514/9.1; 514/9.2; 514/9.9; 514/10.1; 514/10.3; 514/10.8; 514/10.9; 514/11.2; 514/12.4; 514/12.6; 514/14.1; 514/19.3; 514/19.6

(57) ABSTRACT

The present invention relates to the use of inclusion bodies as vehicles for therapeutic protein delivery. This method is applicable to the delivery of therapeutic proteins to intracellular locations. In addition, the invention also relates to the administration of a cell or a pharmaceutical composition comprising inclusion bodies formed by therapeutic proteins. These inclusion bodies formed by therapeutic proteins could be used for the treatment of different diseases.

### BACKGROUND OF THE INVENTION

1. Field of the Invention

The present invention relates in general to the field of disease therapy, and more particularly to the use of inclusion bodies formed by therapeutic proteins for the treatment of different diseases, wherein said treatment does not require the generation of an immune response against said therapeutic protein.

2. Background Art

Without limiting the scope of the invention, its background is described in connection with the use of inclusion bodies as therapeutic agents. The treatment of several mammalian diseases, such as cancer, diabetes, Parkinson's, etc., requires the administration to the subject of therapeutic proteins which can revert the pathological process involved, and, occasionally, said therapeutic proteins must reach the interior of the cell. There are different ways of targeting these therapeutic proteins into the cell. They can be administered in soluble or free form, i.e., without a carrier. However, during the past few years alternative strategies to carry pharmaceutical substances, including peptides and proteins, for the purpose of increasing their stability and internalization have been developed. Micelles, liposomes, nanoparticles (e.g., lipidic, polymeric, metallic, or functionalized nanoparticles), nanocrystals, cyclodextrins, dendrimers, nanotubes, etc. (Marcato & Duran (2008) “New aspects of nanopharmaceutical delivery systems” J. Nanosci. Nanotechnol. (5): 2216-29) stand out among the strategies developed. The main drawbacks of nanoparticle-based strategies are their low specificity and the difficulty in producing them easily and quickly.

In the context of recombinant therapeutic protein production, soluble protein species are generally believed to be properly folded and fully functional. However, recombinant therapeutic proteins expressed in bacteria often accumulate in the bacterial cytoplasm as insoluble aggregates known as inclusion bodies (IBs) (Marston (1986) “The purification of eukaryotic polypeptides synthesized in Escherichia coli” Biochem. J. 240:1-12; Schein (1989) “Production of Soluble Recombinant Proteins in Bacteria” Nature Biotechnology 7:1141-1149). While the exact mechanism for IB formation is not fully understood and may vary with different proteins and expression conditions, it is generally thought that target proteins are being made faster than they can fold into the native structure. If a protein is partially folded or misfolded, it will generally have hydrophobic regions that can interact with other similar proteins and form aggregates. Once in an IB, the protein is usually protected from proteolytic attack and is the predominant protein within the IB.

The resistance of proteins in IBs to proteolysis after oral administration was the basis for the production of an oral vaccine against the liver fluke (see WO 2004/058816). Immunogenic IBs formed by a cysteine protease from Fasciola hepatica were fused to the core protein of the hepatitis type B virus (HBV) and administered orally to rats. It was observed that the administration of IBs was capable of inducing an immune response against the protein in the IB. The observed therapeutic effect was a protective response against an exogenous pathogen mediated by the host's immune system's response against a heterologous immunogen contained in an IB. Thus, the effect of the administered composition was not the result of the proteolytic activity of the cysteine protease.

In contrast, the present invention relates to the use of proteins in inclusion bodies wherein the therapeutic effect of the administered protein is not mediated by the host's immune response. Instead, the therapeutic effect is the direct result of the administered protein performing its specific biological function, for example as an enzyme, chaperone, receptor, cofactor, transporter, cytoskeletal or other structural component, or transcriptional regulator.

IBs can vary from being mostly natively folded proteins which are easily solubilized under mild conditions, to being misfolded, effectively irreversibly insoluble material that requires high concentrations of denaturants to be solubilized. The latter category is by far the most common. For this reason, considerable effort has been devoted to finding conditions under which denatured proteins in IBs can be solubilized and refolded efficiently (see, e.g., Burgess (2009) “Refolding Solubilized Inclusion Body Proteins”. Methods in Enzymology 463: 259-282; Cabrita & Bottomley (2004) “Protein Expression and Refolding—A Practical Guide to Getting the Most Out of Inclusion Bodies”. Biotechnology Annual Review 10: 31-50).

Solubilization and refolding of inclusion body proteins into bioactive forms is cumbersome, results in poor recovery and accounts for the major cost in production of recombinant proteins from prokaryotic sources. It is therefore necessary to develop more cost effective protein production and delivery systems capable of transporting functionally active therapeutic proteins administered orally to the gastrointestinal tract without the drawbacks of the systems known in the art. Preferably, such systems should overexpress the therapeutic protein of interest with a high yield, and deliver it to a therapeutic target without the need for denaturing the protein in order to solubilize it.

Although proteins trapped in insoluble inclusion bodies (IBs) are generally believed to be (i) misfolded and (ii) inactive (Baneyx & Mujacic (2004) “Recombinant protein folding and misfolding in Escherichia coli” Nature Biotechnol. 22:1399-1408), current research no longer supports this assumption. A growing number of studies in the scientific literature describe IBs as entities formed by functional protein species with native secondary structure. Furthermore, the structural and functional diversity of the model proteins used in these studies leaves little room to speculate about these observations being artifacts or peculiarities of certain protein species (Garcia-Fruitós et al. (2005) “Aggregation as bacterial inclusion bodies does not imply inactivation of enzymes and fluorescent proteins” Microb. Cell Fact. 4:27; Jevsevar et al. (2005) “Production of nonclassical inclusion bodies from which correctly folded protein can be extracted.” Biotechnol. Prog. 21:632-639; Garcia-Fruitós et al. (2007) “Localization of Functional Polypeptides in Bacterial Inclusion Bodies” Appl. Environ. Microbiol. 73:289-294). Recent reviews in this area have reported IBs containing properly folded proteins (Doglia et al. (2008) “Fourier transform infrared spectroscopy analysis of the conformational quality of recombinant proteins within inclusion bodies” Biotechnol. J. 3:193-201; Ventura & Villayerde (2006) “Protein quality in bacterial inclusion bodies” Trends Biotechnol. 24:179-185), casting doubt on the paradigm of considering recombinant protein solubility as equivalent to protein conformational quality (Gonzalez-Montalban et al. (2007) “Recombinant protein solubility—does more mean better?” Nat. Biotechnol. 25:718-720).

Since essentially any protein or polypeptide species can be obtained in the form of nanometer to micrometer-sized IBs (Margreiter et al. (2008) “Size characterization of inclusion bodies by sedimentation field-flow fractionation” J. Biotechnol. 138:67-73) and functional proteins or polypeptides are present in IBs, these nanoparticles, spontaneously formed in cell factories, may be used as potential carriers of functional proteins into mammalian cells and organisms, acting as therapeutic nanopills or micropills for protein replacement and other therapies. However, for their potential application to be feasible, IBs should be (i) mechanically stable, (ii) non-toxic to mammalian cells, (iii) capable of interacting with and penetrating target cells while keeping the biological activity of the forming protein, and (iv) capable of producing the desirable physiological effect associated to the biological activity of the carried protein.

### BRIEF SUMMARY OF THE INVENTION

The invention is based, at least in part, on the development of methods for efficiently introducing proteins with therapeutic application inside the cell. The solution provided by the inventors takes advantage of the possibility of overexpressing the therapeutic protein of interest in the form of inclusion bodies (IBs) to introduce said therapeutic protein of interest inside the cell.

IBs generated by the aggregation of a recombinant therapeutic protein are stable and capable of penetrating the cell, as clearly shown in Example 1, which describes the production and characteristics of IBs formed by the aggregation of the green fluorescent protein (GFP). Furthermore, said recombinant protein maintains its biological activity, as illustrated in Example 2, which describes the production of IBs formed by the aggregation of the human Hsp70 chaperone, a potent inhibitor of cell apoptosis. Therefore, said IBs can be used in the treatment of a disease which can improve by administering said therapeutic protein.

In a first aspect, the invention relates to a method to deliver a therapeutic protein to a subject in need thereof comprising administering a non-solubilized inclusion body wherein said inclusion body comprises said therapeutic protein, and wherein said therapeutic protein is not a vaccine immunogen. In some embodiments, the inclusion body is insoluble. In other embodiments, the inclusion body may be internalized by a target cell.

In one aspect of the present invention, the therapeutic protein in the inclusion body is biologically active. However, one of ordinary skill in the art would appreciate that some proteins may require further processing to yield a biologically active form, e.g., a pro-enzyme in an inclusion body might be correctly folded but require to be cleaved by intracellular proteases to yield the active enzyme form.

The therapeutic protein in the inclusion body may be a recombinant protein. In a particular embodiment, said therapeutic protein is erythropoietin (EPO), corticotropin-releasing hormone (CRH), growth hormone-releasing hormone (GHRH), gonadotropin-releasing hormone (GnRH), thyrotropin-releasing hormone (TRH), prolactin-releasing hormone (PRH), melanotropin-releasing hormone (MRH), prolactin-inhibiting hormone (PIH), somatostatin, adrenocorticotropic hormone (ACTH), somatotropin or growth hormone (GH), luteinizing hormone (LH), follicle-stimulating hormone (FSH), thyrotropin (TSH or thyroid-stimulating hormone), prolactin, oxytocin, antidiuretic hormone (ADH or vasopressin), melatonin, Müllerian inhibiting factor, calcitonin, parathyroid hormone, gastrin, cholecystokinin (CCK), secretin, insulin-like growth factor type I (IGF-I), insulin-like growth factor type II (IGF-II), atrial natriuretic peptide (ANP), human chorionic gonadotropin (hCG), insulin, glucagon, somatostatin, pancreatic polypeptide (PP), leptin, neuropeptide Y, renin, angiotensin I, angiotensin II, factor VIII, factor IX, tissue factor, factor VII, factor X, thrombin, factor V, factor XI, factor XIII, interleukin 1 (IL-1), Tumor Necrosis Factor Alpha (TNF-α), interleukin 6 (IL-6), interleukin 8 (IL-8 and chemokines), interleukin 12 (IL-12), interleukin 16 (IL-16), interferons alpha, beta, gamma, nerve growth factor (NGF), platelet-derived growth factor (PDGF), transforming growth factor beta (TGF-β), bone morphogenetic proteins (BMPs), fibroblast growth factors (FGF and KGF), epidermal growth factor (EGF and those related thereto), vascular endothelial growth factor (VEGF), granulocyte colony-stimulating factor (G-CSF), glial growth factor, keratinocyte growth factor, endothelial growth factor, alpha-1 antitrypsin, tumor necrosis factor, granulocyte-macrophage colony-stimulating factor (GM-C SF), cyclosporine, fibrinogen, lactoferrin, tissue-type plasminogen activator (tPA), chymotrypsin, immunoglobins, hirudin, superoxide dismutase, imiglucerase or a chaperone (e.g., Hsp70). In some embodiments, the protein may be administered orally.

The recombinant proteins may be homologous or heterologous proteins, which could be expressed in a suitable expression system, for example bacteria, yeast and other fungi, insect cells, or mammalian cells. The proteins in the IBs could be proteins that are spontaneously deposited in IBs or they could be proteins that require conjugated to an inclusion body-inducing polypeptide. One artisan would recognize that different polypeptides could be fused to the therapeutic protein to induce the formation of IBs. In one particular embodiment, the inclusion body-inducing polypeptide comprises the VP1 pentamer-forming capsid protein of Foot and Mouth Disease Virus (FMDV) or a fragment therein. In some embodiments, the IB-forming protein may be conjugated to a protein tag, such as His6-tag.

In a particular embodiment, the therapeutic protein which aggregates to form said IBs is an anti-apoptotic protein for the treatment of diseases associated with an increase in apoptosis, e.g., a chaperone. In one embodiment of the present invention, said chaperone is Hsp70, and the disease which can improve is a disease in which an anti-apoptotic effect is required, such as cancer, AIDS, Alzheimer's disease, Parkinson's disease, amyotrophic lateral sclerosis, retinitis pigmentosa, cerebellar degeneration, myelodysplastic syndromes, aplastic anemia, myocardial infarction, apoplexy, reperfusion injury, or liver damage. In some embodiments, the cancer may be human leukemia or cervical cancer, and the apoptosis may be caused by chemotherapy with an anticancer agent, such as cisplatin.

In another aspect, the invention refers a cell comprising the therapeutic protein delivered in IB form. Such cell may be an autologous cell or a heterologous cell. Another aspect of the invention is a method for treatment of a disease or condition that benefits from treatment with therapeutic proteins in a patient, the method comprising administering to said patient an effective amount of a therapeutic protein in an IB. In one embodiment of the invention, the method for treatment of a disease or condition comprises administering to a patient an effective amount of a cell containing a therapeutic protein in IB form.

Another embodiment of the invention is a method for treatment of a disease or condition that benefits from treatment with therapeutic proteins in a patient, wherein the method comprises the steps of (a) extracting cells from said patient, (b) contacting said cells with the therapeutic protein in IB form, and (c) implanting said cells containing said therapeutic protein into the patient.

In another aspect, the present invention refers to a pharmaceutical formulation comprising a therapeutic protein in inclusion body form and a pharmacologically acceptable excipient. Still in another aspect, the invention refers to a pharmaceutical formulation comprising a pharmacologically acceptable excipient and a cell, wherein said cell contains a therapeutic protein in inclusion body form. In some embodiments, the cell does not contain IBs when administered; in these embodiments, the cell comprises a polynucleotide sequence encoding a therapeutic protein wherein said therapeutic protein forms IBs upon expression.

### BRIEF DESCRIPTION OF THE DRAWINGS/FIGURES

FIG. 1 shows the morphological and functional characterization of inclusion bodies (IBs). FIG. 1A shows confocal microscopy images of wild type (WT) cells overproducing IBs formed by GFP. Cell samples taken at 1 h, 2 h and 3 h after the induction of IB production are shown. FIG. 1B shows confocal microscopy images using metamorph lookup table of DnaK- and WT cells producing GFP IBs after 2 h of induction of the recombinant gene expression (top), and plain confocal images of IBs purified from these strains after 3 h of induction (bottom). Scale bars in FIG. 1A and FIG. 1B indicate 1 μm.

FIG. 2 shows the stability of GFP IBs. FIG. 2A shows stability in aqueous buffer at 37° C., 25° C. and 4° C., or lyophilized (L) and further stored at 25° C. or 4° C. FIG. 2B shows confocal microscopy images of purified IBs stored in buffer for one month at 25° C., 4° C. and −80° C., and after lyophilization/reconstitution (L).

FIG. 3 shows the interaction between the IBs and the cell. Optical and confocal images of HeLa cells growing in 35 mm polystyrene plates 4 h after exposure to different concentrations (indicated on top) of VP1GFP IBs produced in WT cells are shown. Lateral projections in the first two images indicate the internalization of particular IBs as representative examples.

FIG. 4 shows the effect of IBs treatment on cisplatin-induced apoptosis in HL-60 cells. FIG. 4A shows the effect of different concentrations of Hsp70 or VP1LAC IBs on cell viability. FIG. 4B shows the effect of undiluted Hsp70 or VP1LAC IBs on cell viability when the cells were simultaneously treated (grey bars) or untreated (black bars) with cisplatin (CDDP). FIG. 4C shows the dose-dependent effect of IBs treatment on CDDP-induced apoptosis.

### DETAILED DESCRIPTION OF THE INVENTION

The present invention is directed to the use of inclusion bodies (IBs), generated from the production in a host cell of a recombinant therapeutic protein, as therapeutic agents, in particular, in the treatment of a disease by administering said therapeutic protein. Therefore, in one aspect, the invention refers to methods to deliver a therapeutic protein in inclusion body form, wherein said IBs are generated by the aggregation of a recombinant therapeutic protein produced in a microorganism or cell line producing IBs.

The present invention relates to the use of proteins in inclusion bodies wherein the therapeutic effect of the administered protein is not mediated by the host's immune response. Instead, the therapeutic effect is the direct result of the administered protein performing its specific biological function, for example as an enzyme, chaperone, receptor, cofactor, transporter, cytoskeletal or other structural component, or transcriptional regulator. In some embodiments, the invention relates to pharmaceutical compositions and cells comprising IBs that contain therapeutic proteins.

As used herein, the expression “inclusion body” or “IB” refers to partial or complete deposits of a recombinant protein in the form of insoluble aggregates, when they are produced in a microorganism or in a suitable recombinant cell system. The abbreviation “IB” will generally be used to refer to an inclusion body (singular) and the abbreviation “IBs” to refer to inclusion bodies (plural). IBs normally have a particle size comprised between 0.05 and 1 μm, although said range can vary. In some cases, but not all cases, these IBs may be recognized as bright refractive spots under an optical microscope. An IB is normally formed by the aggregation of an insoluble form of the product of a foreign gene. The foreign gene which is introduced into a plasmid could be a gene encoding a heterologous or homologous protein. It is more likely that a heterologous protein forms IB, since it is a foreign protein for the cell generating it, but a homologous protein can also produce IBs, for example, due to specific sequences of the plasmid promoter increasing the production of said protein. The overexpressed protein is preferably a therapeutic protein.

“Heterologous proteins” are those proteins foreign to the host cell being utilized, such as a human protein produced by E. coli. While the heterologous protein may be prokaryotic or eukaryotic, preferably it is eukaryotic, more preferably mammalian, and most preferably human. In certain embodiments of the invention, it is recombinantly produced (e.g., it is a recombinant polypeptide or a recombinant protein).

IBs can accumulate in the cytoplasm or in the periplasm of prokaryotic cells, depending on whether the recombinant protein has been designed to accumulated in the cytoplasm or to be secreted to the periplasm. A therapeutic protein may be directed to the periplasm of a prokaryotic cell or to a certain cellular compartment in the case of an eukaryotic cell wherein said protein would form inclusion bodies. Protein location may be directed by using a signal sequence. Virtually any signal sequence can be used to put the present invention into practice (e.g., Galliciotti et al. (2001) “Signal-sequence Trap in Mammalian and Yeast Cells: A Comparison” J. Membrane Biology 183:175-182; Stampolidis et al. (2009) “Periplasmically-exported lupanine hydroxylase undergoes transition from soluble to functional inclusion bodies in Escherichia coli.” Arch. Biochem. Biophys. 484:8-15; Mergulhâo & Monteiro (2007) “Periplasmic targeting of recombinant proteins in Escherichia coli.” Methods Mol. Biol. 390:47-61). For example, expressed proteins may be redirected to peroxisomes with a PTS (peroxisomal targeting sequence), to the mitochondrial matrix with a MTS (mitochondrial targeting signal), to the nucleus with a NLS (nuclear localization signal), or to the endoplasmic reticulum with a SRP (signal recognition peptide). Within the context of the present invention, the term “signal peptide” includes targeting signals, signal sequences, transit peptides and localization signals.

The recombinant nucleic acid sequence encoding the overexpressed protein may include an inclusion body fusion partner (i.e., inclusion body-inducing protein, peptide or polypeptide) that is operably linked to the therapeutic protein, e.g. the VP1 protein of the foot and mouth disease virus (FMDV), the F19D mutant of human Ab-amyloid protein, or baculoviral polyhedrin (see, e.g., Li et at (2007) “High and compact formation of baculoviral polyhedron-induced inclusion body by co-expression of baculoviral FP25 in Escherichia coli”. Biotechnol. Bioeng. 96:1183-1190). It is known in the art that linking an inclusion body fusion partner to a preselected polypeptide will cause the tandem polypeptide to form an inclusion body. It is also known in the art that the amino acid sequence of an inclusion body fusion partner can be altered to produce inclusion bodies that exhibit useful characteristics.

The overexpressed protein can typically represent 70-100% of the IB material, which can contain, in small amounts, other proteins (e.g., membrane proteins, etc.), ribosomal components and a small amount of phospholipids and nucleic acids which are adsorbed after cell lysis. Some chaperones or folding modulators (such as DnaK, GroEL and IbpA/B) are sometimes, but not always, associated with IB formation. The present invention shows that IBs are capable of penetrating a cell and being located in a subcellular compartment, wherein the therapeutic protein (the aggregation of which forms the IBs), is active and, therefore, performs its function.

By “therapeutic protein” as used throughout this specification and claims is meant any naturally or non-naturally occurring protein or polypeptide possessing valuable biological properties that may be useful in the treatment of diseases or in preventive medicine by conferring a therapeutic benefit to a host when administered to the host, or when it is expressed in cells of the host. For the purposes of this invention, beneficial or desired clinical results of the therapeutic protein include, but are not limited to, symptom relief, reduction of the extension of the disease, stabilized pathological state (specifically not worsened), delaying or stopping the progression of the disease, improvement or palliation of the pathological state and remission (both partial and total), both detectable and non-detectable. Proteins whose therapeutic effect is exerted through a host's immune response (e.g., a vaccine immunogen) are not considered therapeutic proteins within the scope of the present invention. Instead, for a protein to be considered a therapeutic protein within the scope of the present invention, its beneficial effect must be the result of the administered protein's specific biological function, for example, as an enzyme, chaperone, receptor, cofactor, transporter, cytoskeletal or other structural cellular component, or transcriptional regulator. One of ordinary skill in the art would appreciate that protein biological functions are not limited to those enumerated above.

As used herein “functional” or “biologically active” polypeptides, proteins, and the like refers to molecules with a biologically active conformation. The skilled artisan will recognize that misfolded or partially folded intermediates may have biological activity. Antigenicity is not considered a biological activity.

It has been predicted that as much as 20-40% of human gene constructs will, under normal cell culture conditions, express as inclusion bodies in E. coli (Stevens (2000) “Design of high-throughput methods of protein production for structural biology” Structure Fold. Des. 8:R177-185). In addition, IB formation can be triggered by multiple factors such as higher induction temperatures, cell cultivation without pH control, osmolarity changes, induction modality, choice of promoter, cell density, culture medium, or—in general—any factor affecting the total expression rate of the system. Also, as previously explained, IB formation can be promoted by the fusion of the therapeutic protein of interest to a suitable inclusion body fusion partner such as the VP1 protein. Thus, virtually any therapeutic protein selected for expression could be directed to deposit as an inclusion body.

Virtually any therapeutic protein capable of forming IBs can be used to put the present invention into practice, for example, hormones, cytokines, coagulation factors, etc. Illustrative non-limiting examples of said therapeutic protein include erythropoietin (EPO), corticotropin-releasing hormone (CRH), growth hormone-releasing hormone (GHRH), gonadotropin-releasing hormone (GnRH), thyrotropin-releasing hormone (TRH), prolactin-releasing hormone (PRH), melanotropin-releasing hormone (MRH), prolactin-inhibiting hormone (PIH), somatostatin, adrenocorticotropic hormone (ACTH), somatotropin or growth hormone (GH), luteinizing hormone (LH), follicle-stimulating hormone (FSH), thyrotropin (TSH or thyroid-stimulating hormone), prolactin, oxytocin, antidiuretic hormone (ADH or vasopressin), melatonin, Müllerian inhibiting factor, calcitonin, parathyroid hormone, gastrin, cholecystokinin (CCK), secretin, insulin-like growth factor type I (IGF-I), insulin-like growth factor type II (IGF-II), atrial natriuretic peptide (ANP), human chorionic gonadotropin (hCG), insulin, glucagon, somatostatin, pancreatic polypeptide (PP), leptin, neuropeptide Y, renin, angiotensin I, angiotensin II, factor VIII, factor IX, tissue factor, factor VII, factor X, thrombin, factor V, factor XI, factor XIII, interleukin 1 (IL-1), Tumor Necrosis Factor Alpha (TNF-α), interleukin 6 (IL-6), interleukin 8 (IL-8 and chemokines), interleukin 12 (IL-12), interleukin 16 (IL-16), interferons alpha, beta, gamma, nerve growth factor (NGF), platelet-derived growth factor (PDGF), transforming growth factor beta (TGF-β), bone morphogenetic proteins (BMPs), fibroblast growth factors (FGF and KGF), epidermal growth factor (EGF and those related thereto), vascular endothelial growth factor (VEGF), granulocyte colony-stimulating factor (G-CSF), glial growth factor, keratinocyte growth factor, endothelial growth factor, alpha-1 antitrypsin, tumor necrosis factor, granulocyte-macrophage colony-stimulating factor (GM-C SF), cyclosporine, fibrinogen, lactoferrin, tissue-type plasminogen activator (tPA), chymotrypsin, immunoglobins, hirudin, superoxide dismutase, imiglucerase, chaperones, etc. In a particular embodiment, the therapeutic protein the aggregation of which forms the IBs of the invention is the human Hsp70 chaperone, a potent inhibitor of cell apoptosis.

The IBs of the invention can be obtained by conventional methods which generally comprise introducing the sequence of nucleic acids encoding the therapeutic protein of interest in a suitable expression system which can produce IBs and culturing it under conditions suitable for the production of said IBs.

It is known in the art that different microorganisms and cell lines produce or have the capacity to produce IBs. Illustrative non-limiting examples of said microorganisms and cell lines producing IBs include, although they are not exclusively limited to, bacteria (Parente et al. (1991) “Prochymosin expression in Bacillus subtilis.” FEMS Microbiol. Lett. 77:243-249), yeasts (Cousins et al. (1987) “High Level Expression of Proinsulin in yeast, S. cerevesiae” Gene 61:256) and insect cell lines (Thomas et al. (1990) “Synthesis of bluetongue virus-encoded phosphoprotein and formation of inclusion bodies by recombinant baculovirus in insect cells: it binds the single-stranded RNA species” J. Gen. Virol. 71:2073-83). There is also the possibility that they are generated in mammalian cell lines (see, e.g., Ioannou et al. (1992) “Overexpression of human alpha-galactosidase A results in its intracellular aggregation, crystallization in lysosomes, and selective secretion” J. Cell Biol. 119:1137-50).

The “bacteria” for the purposes herein include eubacteria and archaebacteria. In certain embodiments of the invention, eubacteria, including gram-positive and gram-negative bacteria, are used in the methods described herein. In one embodiment of the invention, gram-negative bacteria are used, e.g. Enterobacteriaceae. Examples of bacteria belonging to Enterobacteriaceae include Escherichia, Enterobacter, Erwinia, Klebsiella, Proteus, Salmonella, Serratia, and Shigella. In one embodiment of the invention, E. coli is used. MC4100, DnaK, and BL21 are suitable E. coli strains used in some embodiments of the present invention. Other suitable E. coli strains include E. coli LG1522 (ATCC No.: BAA-1907™), E. coli AMC 198 (ATCC No.: CRM-11229™), E. coli Crooks (ATCC No.: CRM-8739™), DH5α, BL26, HB101, JM107, D21, JM103, AB 1157, and in general any of the strains available at the Yale University Coli Genetic Stock Center or other repositories (Maloy & Hughes (2007) “Strain collections and genetic nomenclature.” Methods Enzymol. 421:3-8).

In some embodiments, gram-positive bacteria are used, e.g. Lactobacillales. Examples of bacteria belonging to the order Lactobacillales include Lactococcus, Lactobacillus, Pediococcus, Oenococcus, Leuconostoc, Enterococcus, and Streptococcus (see, e.g., Ljungh & Wadstrom (2009) “Lactobacillus Molecular Biology: From Genomics to Probiotics,” Horizon Scientific Press, ISBN 1904455417; Charalampopoulos & Rastall (2009) “Prebiotics and Probiotics Science and Technology,” Springer ISBN 0387790578). These examples are illustrative rather than limiting. Mutant cells of any of the above-mentioned bacteria may also be employed. It is, of course, necessary to select the appropriate bacteria taking into consideration replicability of the replicon in the cells of a bacterium. For example, E. coli, Serratia, or Salmonella species can be suitably used as the host when well-known plasmids such as pBR322, pBR325, pACYC177, or pKN410 or other commercially available vectors are used to supply the replicon.

It is known in the art that Lactobacillales can effectively deliver to the intestine therapies for the treatment of intestinal inflammatory diseases such as ulcerative colitis (see, e.g. Nishitani et al. (2009) “Lactobacillus lactis subsp. cremoris FC alleviates symptoms of colitis induced by dextran sulfate sodium in mice.” Int. Immunopharmacol. 9(12):1444-51; Vandenbroucke et al. (2010) “Orally administered L. lactis secreting an anti-TNF nanobody demonstrate efficacy in chronic colitis.” Mucosal Immunol. 3(1):49-56).

In some embodiments, fungi may be used, e.g., Geotrichum candidum, Kluveromyces marxianus, and Pichia fermentans. Fungi have also been described as effective vehicles to deliver to the intestine therapies for the treatment of intestinal inflammatory diseases such as ulcerative colitis (see, e.g., Lavi et al. (2010) “Orally administered glucans from the edible mushroom Pleurotus pulmonarius reduce acute inflammation in dextran sulfate sodium-induced experimental colitis”. Br. J. Nutr. 103(3):393-402).

As used herein, the expressions “cell,” “cell line,” “strain,” and “cell culture” are used interchangeably and all such designations include progeny. Thus, when a nucleic acid encoding a therapeutic protein is introduced in a “cell” or “cell line,” the term includes the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included.

The introduction of the sequence encoding the therapeutic protein in said microorganisms and cell lines is carried out by means of conventional methods. In brief, expression vectors capable of autonomous replication and protein expression relative to the host cell genome are introduced into the host cell. Construction of appropriate expression vectors is well known in the art. See, e.g., Sambrook et al. (2001) “Molecular Cloning, A Laboratory Manual”, Cold Spring Harbor Laboratory Press (Cold Spring Harbor, N.Y.); Ausubel et al. (1994) “Current Protocols in Molecular Biology” (New York: Greene Publishing Associates and Wiley-Interscience); and Baneyx (1999) “Recombinant protein expression in Escherichia coli” Current Opinion in Biotechnology 10:411-421.

Appropriate prokaryotic cells, including bacteria, and expression vectors are available commercially through, for example, the American Type Culture Collection (ATCC, Rockville, Md.). Methods for the large scale growth of prokaryotic cells, and especially bacterial cell culture are well known in the art and these methods can be used in the context of the invention. In some embodiments of the present invention, the pTVP1GFP (Apr), pReceiver-MO2, and pReceiver-B01 vectors (Genecopoeia, Rockville, Md.) were used.

For example, prokaryotic host cells are tranfected with expression or cloning vectors encoding the recombinant therapeutic protein of interest and cultured in conventional nutrient media modified as appropriate for inducing promoters, selecting transformants, or amplifying the genes encoding the desired sequences. The nucleic acid encoding the therapeutic protein of interest is suitably RNA, cDNA, or genomic DNA from any source, provided it encodes the polypeptide(s) of interest. Methods are well known for selecting the appropriate nucleic acid for expression of polypeptides and proteins (including variants thereof) in microbial hosts. Nucleic acid molecules encoding the therapeutic protein of interest are prepared by a variety of methods known in the art. For example, a DNA encoding Hsp70 may be isolated and sequenced, e.g., by using oligonucleotide probes that are capable of binding specifically to the gene encoding Hsp70.

The nucleic acid (e.g, cDNA or genomic DNA) encoding the therapeutic protein is suitably inserted into a replicable vector for expression in the microorganism under the control of a suitable promoter. Many vectors are available for this purpose, and selection of the appropriate vector will depend mainly on the size of the nucleic acid to be inserted into the vector and the particular host cell to be transformed with the vector. Each vector contains various components depending on the particular host cell with which it is compatible. Depending on the particular type of host, the vector components generally include, but are not limited to, one or more of the following: a signal sequence, an origin of replication, one or more marker genes, a promoter, and a transcription termination sequence.

In general, plasmid vectors containing replicon and control sequences that are derived from species compatible with the host cell are used in connection with microbial hosts. The vector ordinarily carries a replication site, as well as marking sequences that are capable of providing phenotypic selection in transformed cells.

(i) Signal Sequence. Therapeutic proteins may be produced recombinantly not only directly, but also as a fusion polypeptide with a heterologous polypeptide, which is typically a signal sequence or other polypeptide having a specific cleavage site at the N-terminus of the mature protein or polypeptide. The signal sequence selected typically is one that is recognized and processed (i.e., cleaved by a signal peptidase) by the host cell. For prokaryotic host cells that do not recognize and process a heterologous polypeptide signal sequence, the signal sequence may be substituted by a prokaryotic signal sequence selected, for example, from the group of the alkaline phosphatase, penicillinase, 1 pp, or heat-stable enterotoxin II leaders.

(ii) Origin of Replication Component. Expression vectors contain a nucleic acid sequence that enables the vector to replicate in one or more selected host cells. Such sequences are well known in the art for a variety of microbes.

(iii) Selection Gene Component. Expression vectors generally contain a selection gene, also termed a selectable marker. This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies other than those caused by the presence of the genetic marker(s), or (c) supply critical nutrients not available from complex media, e.g., the gene encoding D-alanine racemase for Bacilli.

One example of a selection scheme utilizes a drug to arrest growth of a host cell. In this case, those cells that are successfully transformed with the nucleic acid of interest produce a polypeptide conferring drug resistance and thus survive the selection regimen. Examples of such dominant selection use the drugs neomycin (Southern & Berg (1982) “Transformation of mammalian cells to antibiotic resistance with a bacterial gene under control of the SV40 early region promoter.” J. Mol. Appl. Genet. 1: 327-341), mycophenolic acid (Mulligan & Berg (1980) “Expression of a bacterial gene in mammalian cells.” Science 209:1422-27) or hygromycin (Sugden et al. (1985) “A vector that replicates as a plasmid and can be efficiently selected in B-lymphoblasts transformed by Epstein-Barr virus” Mol. Cell. Biol. 5:410-413). The three examples given above employ bacterial genes under eukaryotic control to convey resistance to the appropriate drug G418 or neomycin (geneticin), xgpt (mycophenolic acid), or hygromycin, respectively.

(iv) Promoter Component. The expression vector for producing the recombinant therapeutic protein of interest contains a suitable promoter that is recognized by the host organism and is operably linked to the nucleic acid encoding the therapeutic protein of interest. Promoters suitable for use with prokaryotic hosts include the beta-lactamase and lactose promoter systems (Chang et al. (1978) “Phenotypic expression in E. coli of a DNA sequence coding for mouse dihydrofolate reductase.” Nature 275:617-624; Goeddel et al. (1979) “Direct expression in Escherichia coli of a DNA sequence coding for human growth hormone” Nature 281:544-48), the arabinose promoter system (Guzman et al. (1992) “FtsL, an essential cytoplasmic membrane protein involved in cell division in Escherichia coli” J. Bacteriol. 174: 7716-7728), alkaline phosphatase, a tryptophan (trp) promoter system (Goeddel et al. (1980) “Synthesis of human fibroblast interferon by E. coli” Nucleic Acids Res. 8: 4057-74, and European Patent EP 36,776) and hybrid promoters such as the tac promoter (De Boer et al. (1983) “The tac promoter: a functional hybrid derived from the trp and lac promoters” Proc. Natl. Acad. Sci. USA 80: 21-25). In one embodiment of the present invention, the recombinant genes were expressed under the control of an isopropyl beta-D-1-thiogalactopyranoside (IPTG) inducible trc (trp-lac) promoter (Egon et al. (1983) “Vectors Bearing a Hybrid trp-lac Promoter Useful for Regulated Expression of Cloned Genes in Escherichia coli” Gene 25:167-178). However, other known bacterial promoters are suitable. Their nucleotide sequences have been published, thereby enabling a skilled worker operably to ligate them to DNA encoding the polypeptide of interest (Siebenlist et al. (1980) “E. coli RNA polymerase interacts homologously with two different promoters.” Cell 20:269-81) using linkers or adaptors to supply any required restriction sites. See also, e.g., Sambrook et al., supra; and Ausubel et al., supra.

Promoters for use in bacterial systems also generally contain a Shine-Dalgarno (S.D.) sequence operably linked to the DNA encoding the polypeptide of interest. The promoter can be removed from the bacterial source DNA by restriction enzyme digestion and inserted into the vector containing the desired DNA.

(v) Construction and Analysis of Vectors. Construction of suitable vectors containing one or more of the above-listed components employs standard ligation techniques. Isolated plasmids or DNA fragments are cleaved, tailored, and re-ligated in the form desired to generate the plasmids required. For analysis to confirm correct sequences in plasmids constructed, successful transformants are selected by antibiotic resistance. Plasmids from the transformants are prepared, analyzed by restriction endonuclease digestion, and/or sequenced by the method of Sanger et al. (Sanger et al. (1977) “DNA sequencing with chain-terminating inhibitors” Proc. Natl. Acad. Sci. USA 74:5463-5467) or Messing et al. (Messing et al. (1981) “A system for shotgun DNA sequencing” Nucleic Acids Res. 9:309-21), or by the method of Maxam & Gilbert (Maxam & Gilbert (1980) “Sequencing end-labeled DNA with base-specific chemical cleavages.” Methods in Enzymology 65:499-560). See also, e.g., Sambrook et al., supra; and Ausubel et al., supra.

The nucleic acid encoding the recombinant therapeutic protein of interest is inserted into the host cells. Typically, this is accomplished by transforming the host cells with the above-described expression vectors and culturing in conventional nutrient media modified as appropriate for inducing the various promoters.

(vi) Culturing the Host Cells. As previously discussed, suitable cells for the practice of the invention are well known in the art. Host cells that express the recombinant therapeutic protein abundantly in the form of inclusion bodies or in the perplasmic or intracellular space are typically used. Prokaryotic cells used to produce the therapeutic protein are grown in media known in the art and suitable for culture of the selected host cells, including the media generally described by Sambrook et al., Molecular Cloning, A Laboratory Manual, Cold Spring Harbor Laboratory Press (Cold Spring Harbor, N.Y.) (2001). Media that are suitable for bacteria include, but are not limited to, Luria-Bertani (LB) broth, AP5 medium, nutrient broth, Neidhardt's minimal medium, and C.R.A.P. minimal or complete medium, plus necessary nutrient supplements. In certain embodiments, the media also contains a selection agent, chosen based on the construction of the expression vector, to selectively permit growth of prokaryotic cells containing the expression vector. For example, ampicillin is added to media for growth of cells expressing ampicillin resistant gene. Any necessary supplements besides carbon, nitrogen, and inorganic phosphate sources may also be included at appropriate concentrations introduced alone or as a mixture with another supplement or medium such as a complex nitrogen source. Optionally the culture medium may contain one or more reducing agents selected from the group consisting of glutathione, cysteine, cystamine, thioglycollate, dithioerythritol, and dithiothreitol.

The prokaryotic host cells are cultured at suitable temperatures. For E. coli growth, for example, the temperature ranges from, e.g., about 20° C. to about 39° C., or from about 25° C. to about 37° C., or at about 30° C.

If the promoter is an inducible promoter, for induction to occur, typically the cells are cultured until a certain optical density is achieved, e.g., a A₅₅₀ of about 200 using a high cell density process, at which point induction is initiated (e.g., by addition of an inducer, by depletion of a medium component, etc.), to induce expression of the gene encoding the polypeptide of interest.

Any necessary supplements may also be included at appropriate concentrations that would be known to those skilled in the art, introduced alone or as a mixture with another supplement or medium such as a complex nitrogen source. The pH of the medium may be any pH from about 5-9, depending mainly on the host organism. For E. coli, the pH is, e.g., from about 6.8 to about 7.4, or about 7.0.

IBs can be isolated from host cells expressing the therapeutic protein by any of a number of art standard techniques. For example, the insoluble recombinant protein is isolated in a suitable isolation buffer by exposing the cells to a buffer of suitable ionic strength to solubilize most host proteins, but in which the subject protein is substantially insoluble, or disrupting the cells so as to release the inclusion bodies from the periplasmic or intracellular space and make them available for recovery by, for example, centrifugation. This technique is well known and is described in, for example, U.S. Pat. No. 4,511,503. Kleid et al., disclose purification of IBs by homogenization followed by centrifugation (Kleid et al. (1984) in “Developments in Industrial Microbiology,” (Society for Industrial Microbiology, Arlington, Va.) 23:217-235). See also, e.g., Fischer et al. (1993) Biotechnology and Bioengineering 41:3-13.

U.S. Pat. No. 5,410,026 describes a typical method for recovering protein from IBs and is summarized as follows. The prokaryotic cells are suspended in a suitable buffer. Typically the buffer consists of a buffering agent suitable for buffering between pH 5 to 9, or about 6 to 8 and a salt. Any suitable salt, including NaCl, is useful to maintain a sufficient ionic strength in the buffered solution. Typically, an ionic strength of about 0.01 to 2 M, or 0.1 to 0.2 M is employed. The cells, while suspended in this bufferm are disrupted or lysed using techniques commonly employed such as, for example, mechanical methods, e.g. homogeneizer (Manton-Gaulin press, Microfluidizer, or Niro-Soavi), a French press, a bead mill, or a sonic disruptor (probe or bath), or by chemical or enzymatic methods.

Examples of chemical or enzymatic methods of cell disruption include spheroplasting, which entails the use of lysozyme to lyse the bacterial wall (Neu & Heppel (1964) “On the surface localization of enzymes in E. coli” Biochem. Biophys. Res. Comm. 17:215-19), and osmotic shock, which involves treatment of viable cells with a solution of high tonicity and with a cold-water wash of low tonicity to release the polypeptides (Neu & Heppel (1965) “The release of enzymes from Escherichia coli by osmotic shock and during the formation of spheroplasts” J. Biol. Chem. 240:3685-3692). Sonication is generally used for disruption of bacteria contained in analytical scale volumes of fermentation broth. At larger scales, high pressure homogenization is typically used.

After the cells are disrupted, the suspension is typically centrifuged at low speed, generally around 500 to 25,000×g, e.g., in one embodiment of the invention about 15,000×g is used, in a standard centrifuge for a time sufficient to pellet substantially all of the insoluble protein. Such times can be simply determined and depend on the volume being centrifuged as well as the centrifuge design. Typically about 10 minutes to 0.5 hours is sufficient to pellet the IBs. In one embodiment of the present invention, the suspension is centrifuged at 15,000×g for 15 minutes.

The resulting pellet contains substantially all of the IBs. If the cell disruption process is not complete, the pellet may also contain intact cells or broken cell fragments. Completeness of cell disruption can be assayed by resuspending the pellet in a small amount of the same buffer solution and examining the suspension with a phase contrast microscope. The presence of broken cell fragments or whole cells indicates that further sonication or other means of disruption is necessary to remove the fragments or cells and other contaminants. After such further disruption, if required, the suspension can be again centrifuged and the pellet recovered, resuspended and reexamined. The process can be repeated until visual examination reveals the absence of broken cell fragments in the pelleted material or until further treatment fails to reduce the size of the resulting pellet.

The above described process can be employed whether the IBs are intracellular or in the periplasmic space. In one embodiment of the invention, the conditions given herein for isolating IBs are directed to IBs containing GFP fused to the amino terminus of VP1 and to IBs containing the Hsp70 chaperon. However, the processes and procedures are thought to be applicable to recombinant proteins in general with minor modifications. In certain embodiments of the invention, the processes and procedures are applicable to manufacturing or industrial scale production and purification of IBS containing a therapeutic protein.

It is known in the art that insoluble therapeutic proteins in IBs can be recovered in biologically active forms by solubilizing or diluting the IBs and refolding the protein (see, e.g., Burgess (2009) “Refolding Solubilized Inclusion Body Proteins.” Methods in Enzymology 463:259-282; Cabrita & Bottomley (2004) “Protein Expression and Refolding—A Practical Guide to Getting the Most Out of Inclusion Bodies.” Biotechnology Annual Review 10:31-50). These solubilized and refolded proteins can then be administered for therapeutic uses. The present invention differs from the art in that the therapeutic proteins of interest are administered in IB form. Thus, expensive and time consuming solubilization and refolding steps are necessary.

The term “treat” or “treatment” designates a therapeutic treatment, in which the object is to stop (reduce) an unwanted physiological change or disorder, such as, for example, the unwanted cell death. For the purposes of this invention, beneficial or desired clinical results include, but are not limited to, symptom relief, reduction of the extension of the disease, stabilized pathological state (specifically not worsened), delaying or stopping the progression of the disease, improvement or palliation of the pathological state and remission (both partial as total), both detectable as non-detectable, as well as prolonging the survival of the treated subject in comparison with the survival expected if treatment is not received. The subjects in need of treatment include subjects who already suffer from the complaint or disorder and the subjects with a tendency to suffer from the complaint or disorder and the subjects whose complaint or disorder has to be prevented. Said treatment does not require the generation of an immune response against said therapeutic protein.

As used herein, the term “disease which can improve by administering a (said) therapeutic protein” refers to a disease which can be treated with the IBs of the invention, containing a therapeutic protein the administration of which improves the disease to be treated; i.e., beneficial clinical results are obtained by means of administering the suitable IBs of the invention. Illustrative non-limiting examples of said diseases include, although they are not limited to, diseases which can be treated with said protein, including cancer, diabetes, etc, as well as monogenic diseases caused by the lack of a protein, including cystic fibrosis and all the deficiencies treated experimentally with gene therapy. Diseases associated with an increase of apoptosis, i.e., with a reduction of cell proliferation, such as AIDS, etc.; neurodegenerative diseases, e.g., Alzheimer's disease, Parkinson's disease, amyotrophic lateral sclerosis, retinitis pigmentosa, cerebellar degeneration, etc.; myelodysplastic syndromes, e.g., aplastic anemia, etc.; ischemic injury, e.g., myocardial infarction, apoplexy, etc.; reperfusion injury; liver damage, e.g., liver damage caused by alcohol, etc. could also be treated.

The IBs of the invention, if desired, can be introduced in cells. Therefore, in another aspect, the invention refers to a cell, hereinafter cell of the invention, comprising an inclusion body of the invention therein. Thus, said cells of the invention include not only the microorganism or cell line producing the IBs of the invention but also target cells internalizing the IBs of the invention. Said cell of the invention can be a prokaryotic or eukaryotic cell. In the case of IBs included inside eukaryotic cells, they could be administered to a subject in need of treatment with autologous cells of said subject containing inclusion bodies of the invention with the suitable therapeutic protein therein, which could be in contact with the IBs of the invention and could subsequently be reimplanted in the subject.

In another aspect, the invention refers to a pharmaceutical composition comprising a therapeutically effective amount of an inclusion body of the invention and a pharmaceutically acceptable carrier for the treatment of diseases which do not require the generation of an immune response against the therapeutic protein forming the IB of the invention. In another aspect, the invention refers to a pharmaceutical composition comprising a therapeutically effective amount of a cell of the invention and a pharmaceutically acceptable carrier for the treatment of diseases which do not require the generation of an immune response against the therapeutic protein forming the IB of the invention.

In some embodiments, the cells administered alone or as part of pharmaceutical compositions contain IBs prior to administration. In other embodiments, the IBs may be generated in situ after administration of the cells expressing the therapeutic protein of interest, e.g. cells transfected with a IB-forming therapeutic protein might develop in the intestine, and IBs might be released upon lysis of the cells in the intestinal lumen.

Examples of diseases which can be treated with the pharmaceutical composition of the invention have been previously mentioned in the present specification.

In the context of the present invention, “therapeutically effective amount” means the amount of inclusion bodies of the invention or of cells of the invention necessary for achieving the desired effect which, in this particular case, is the treatment of diseases which can be treated with the therapeutic protein contained in the IBs of the invention. The therapeutically effective amount of the IB of the invention or of the cell of the invention to be administered will generally depend, among other factors, on the individual who is to be treated, on the severity of the disease that said individual suffers from, on the form of administration chosen, etc.

The pharmaceutical composition of the invention can be administered by any suitable route of administration, for example, oral, by inhalation, parenteral (for example, subcutaneous, intraperitoneal, intravenous, intramuscular route, etc.), rectal route, etc.

Illustrative examples of dosage forms for administration by the oral route include tablets, capsules, granulates, solutions, suspensions, etc., and can contain the conventional excipients, such as binders, diluents, disintegrants, lubricants, wetting agents, etc., and can be prepared by conventional methods. The pharmaceutical compositions can also be adapted for their parenteral administration, in the form of, for example, sterile solutions, suspensions or lyophilized products, in the suitable dosage form; in this case, said pharmaceutical compositions will include the suitable excipients, such as buffers, surfactants, etc. In any case, the excipients will be chosen according to the selected pharmaceutical dosage form.

A review of the different dosage forms for the administration of drugs and their preparation can be found in the book “Tratado de Farmacia Galénica”, by C. Faulí i Trillo, 10 Edition, 1993, Luzan 5, S. A. de Ediciones.

In another aspect, the invention relates to a method of treatment of a disease which can be improved by a therapeutic protein comprising administering to a subject in need of treatment a therapeutically effective amount of IBs of the invention, wherein said treatment does not require the generation of an immune response against said therapeutic protein.

In another aspect, the invention relates to a method of treatment of a disease which can be improved by a therapeutic protein, wherein said treatment does not require the generation of an immune response against said therapeutic protein, comprising administering to a subject in need of treatment autologous cells of said subject containing inclusion bodies of the invention with the suitable therapeutic protein therein. Briefly, to put said method of treatment into practice, cells are extracted from the subject to be treated, they are contacted with IBs of the invention containing the suitable therapeutic protein under conditions which allow internalizing said IBs of the invention in said cells, and, subsequently, they are implanted by conventional methods in the subject to be treated. The therapeutic protein present in the IBs of the invention can produce the desired effect by different mechanisms, for example, replacing a deficient protein, or exerting their effect on other protein or on said cells, etc. The IBs of the invention can likewise be used for an in vivo method of treatment, wherein the IB of the invention would be administered to a patient with a disease which can be treated with said IBs.

The following examples illustrate the invention and must not be considered as limiting the scope thereof.

### Example 1

### Production and Characterization of IBs by Aggregation of GFP

This assay was performed to analyze if IBs produced in bacteria could be internalized by eukaryotic cells and if said internalization could inactivate the biological activity of the protein. To carry out this example IBs were produced based on the green fluorescent protein (GFP).

### Materials and Methods

1.1 Production of Inclusion Bodies—Inclusion bodies (IBs) were produced in Escherichia coli MC4100 strains (WT regarding protein folding and degradation, araD139 Δ(argF-lac) U169 rpsL150 relA1 flbB5301 deoC1 ptsF25 rbsR) and in a strain derived thereof, JGT20 (deficient in the main chaperone DnaK, dnak756 thr::Tn10), hereinafter DnaK strain. These strains were transformed with the expression vector pTVP1GFP (ApR) (Garcia-Fruitós et al. (2005) “Aggregation as bacterial inclusion bodies does not imply inactivation of enzymes and fluorescent proteins” Microb. Cell. Fact. 4:27), encoding the green fluorescent protein (GFP) fused at the amino terminus to VP1, the pentamer-forming capsid protein of Foot and Mouth Disease Virus (FMDV) (Gonzalez-Montalban et al. (2007) “Amyloid-linked cellular toxicity triggered by bacterial inclusion bodies” Biochem. Biophys. Res. Commun. 355:637-642). This viral protein, being highly hydrophobic, directs the deposition of fusion proteins as inclusion bodies (Doglia et al. (2008) “Fourier transform infrared spectroscopy analysis of the conformational quality of recombinant proteins within inclusion bodies” Biotechnol. J. 3:193-201). A similar construct, VP1LAC, encodes a previously described beta-galactosidase fusion (Garcia-Fruitós et al. (2005) “Aggregation as bacterial inclusion bodies does not imply inactivation of enzymes and fluorescent proteins” Microb. Cell. Fact. 4: 27). The recombinant genes were expressed under the control of an isopropyl beta-D-1-thiogalactopyranoside (IPTG) inducible-trc promoter. The bacteria were cultured in Luria Bertani (LB) rich medium (Sigma-Aldrich, 28760 Madrid, Spain), supplemented with 100 μg/ml of ampicillin, and the recombinant gene expression was induced by adding 1 mM IPTG. IBs were detectable after 1 h of IPTG addition.

1.2 Purification of the Inclusion Bodies (IBs)—Samples of 200 ml of bacterial cultures were centrifuged at 4° C. at 5.000 g for 5 minutes and resuspended in 50 ml of lysis buffer (50 mM TrisHCl pH 8.1, 100 mM NaCl and 1 mM EDTA). Ice jacketed samples were sonicated using a Braun LabsonicU probe sonicator (Braun Biotech International) for 25 to 40 minutes, at 40% of amplitude under 0.5 s cycles. Once sonicated, 28 μl of 100 mM phenylmethanesulphonylfluoride (PMSF) and 23 μl of lysozime were added to samples that were incubated at 37° C. under agitation for 45 min. After that, 40 μl of Nonidet P40 (NP-40) were added and the mixture was kept for 1 h at 4° C. under agitation. DNA was removed with 120 μl of 1 mg/ml DNase and 120 μl of 1 M Mg₂SO₄ for 45 min at 37° C. under agitation. Finally, samples were centrifuged at 4° C. at 15000 g for 15 min and the pellet, containing pure IBs, was washed with lysis buffer containing 0.5% Triton X-100 and stored at −20° C. until analysis.

1.3 Microscopic Analysis of the Bacteria and the IBs—Samples were analyzed by using a Leica TSC SP2 AOBS confocal fluorescence microscope (Leica Microsystems Heidelberg GmbH, Manheim, Germany) after excitation at 488 nm, and images were recorded at emission wavelengths between 500 and 600 nm (63× (NA 1.4 oil) using a Plan Apochromat objective (zoom 8; 1,024 by 1,024 pixels). For the analysis of bacterial cells producing fluorescent IBs, samples taken 1, 2 or 3 h after IPTG induction were fixed with 0.2% formaldehyde in phosphate buffered saline (PBS) and stored at 4° C. until their use. Isolated IBs were resuspended in 20 ml of PBS.

1.4 Stability Analyses—IBs obtained in DnaK-cells for 5 h were diluted in PBS with 10 g/l bovine serum albumin (BSA) and 60 g/l sucrose, in the presence of gentamicin at 40 mg/l, penicillin at 100 U/ml and streptomycin at 10 μg/ml, and aliquots incubated at different temperatures (37° C., 25° C. or 4° C.). At different times, samples were frozen at −80° C. until fluorescence determination. Fluorescence was analyzed in a Cary Eclipse fluorescence spectrophotometer (Variant, Inc., Palo Alto, Calif.) by using an excitation wavelength of 450 nm and detecting the fluorescence emission at 510 nm. Results are referred to as the percentage of remaining activity or fluorescence with respect to control samples kept at −80° C., that were fully stable. Another set of samples was lyophilized in a Cryodos-80 lyophilizer, from Telstar (Terrassa, Spain) and stored at either 4° C. or 25° C. until analysis.

1.5 Confocal laser scanning microscopy—HeLa (cervical cancer cell line; ATCC: CCL-2™) and NIH3T3 (fibroblast cell line; ATCC: CRL-1658™) cell cultures were seeded at a density of 70% on glass plates (MatTek Corporation, Ashland, Mass., USA) 24 h before adding VP1GFP inclusion bodies at different concentrations: 2 μM, 5 μM and 10 μM. Four hours after adding the IBs, the living cells were examined using a spectral confocal Leica TCS SP5 AOBS (Leica Microsystems, Mannheim, Germany) using a Plan Apochromat lens (63×, N.A. 1.4 oil). For nuclear and plasma membrane labeling, cells were incubated with 5 μg/ml of Hoechst 33342 and 5 μg/ml of CellMask (both from Molecular Probes, Inc., Eugene, Oreg., USA) respectively for 5 min at room temperature, and washed twice prior to confocal detection. Nuclei were excited with 405 nm diode laser beam, and detected at 414-461 nm (blue channel); plasma membrane was detected by exciting with the light of a 633 nm helium neon laser and fluorescence was detected at 656-789 nm (far red channel); finally, Argon laser 488-nm line was used for imaging VP1GFP IBs (green channel, emission=500-537 nm).

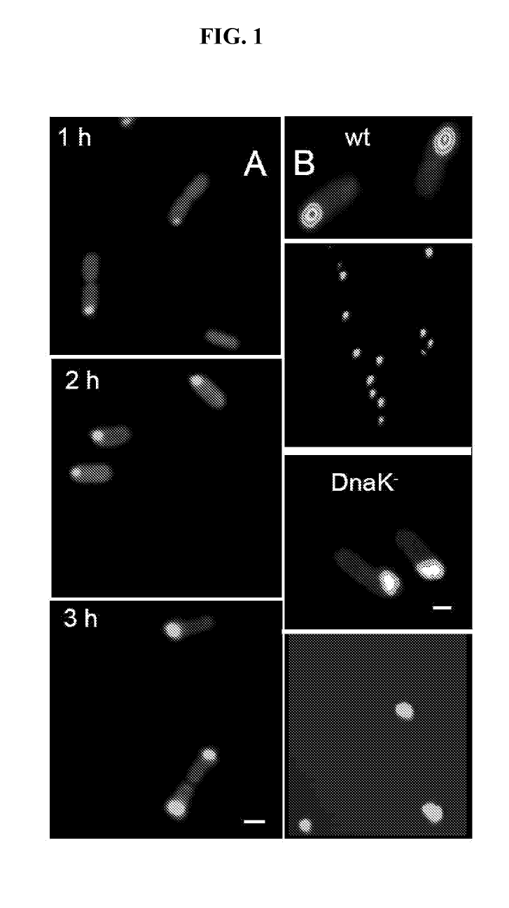

RESULTS—GFP-based inclusion bodies (IBs) have been shown to be excellent models to verify the conformational status and biological activity of the embedded proteins (Garcia-Fruitós et al. (2005) “Aggregation as bacterial inclusion bodies does not imply inactivation of enzymes and fluorescent proteins” Microb. Cell. Fact. 4:27; Garcia-Fruitós et al. (2007) “Localization of functional polypeptides in bacterial inclusion bodies” Appl. Environ. Microbiol. 73:289-294). Therefore, the inventors chose this model to evaluate the mechanical and functional stability of bacterial IBs, during and after purification. The growth of these particles in bacteria producing a recombinant GFP (VP1GFP) is shown in the images in FIG. 1A. The images show a defined growth pattern, with a regular volumetric growth.

FIG. 1B shows microscopy confocal images indicating the location of the fluorescence of IBs produced in WT bacteria or in mutants lacking the chaperone DnaK. IBs are larger in the mutant cells. The corresponding confocal images of IBs after purification are presented in the panels immediately below each one of the panels showing the cells. These images show the high mechanical stability of IBs after being exposed to intense mechanical stress during purification, including ultrasonic disruption of the cells and centrifugation.

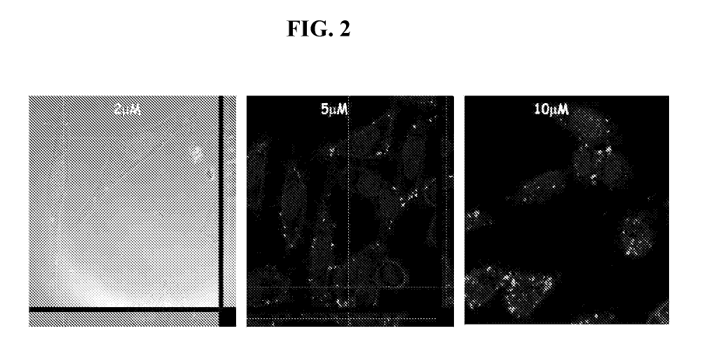

To further confirm the stability of IBs, especially regarding the biological activity of the protein included therein, fluorescence of VP1GFP IBs was monitored in different storage conditions. Freezing and lyophilization, which are storage conditions commonly used for biological materials were selected. As observed in FIG. 2A, IBs were fully functionally stable during long periods of time even when stored at 37° C., supporting their potential use under physiological conditions, both in cell cultures and in whole organisms, since the temperature did not compromise biological activity. Furthermore, the morphology of IBs was unchanged under all these conditions (FIG. 2B), indicative again of a robust mechanical stability.

At this stage, it was decided to test whether IBs could interact with cultured mammalian cells and especially, if this interaction could inactivate the biological activity of the protein, monitored by the fluorescence of a VP1 GFP IB, since neither the stability nor the biological activity of IBs during such interaction had ever been analyzed.

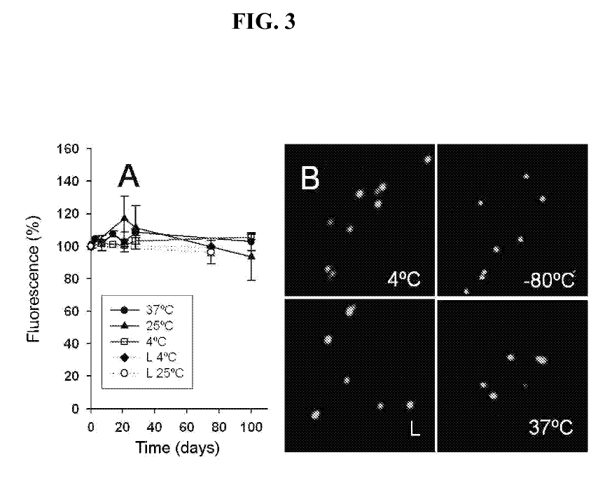

FIG. 3 shows cultured HeLa cells 4 h after exposure at different concentrations of IBs (2, 5 and 10 μM), analyzed under conventional optical microscopy (2 μM) or confocal microscopy with cell staining (5 and 10 μM). At these doses, cells were completely healthy showing no cytopathic effects, while IBs appeared as intimately interacting with cell membranes, and some of them clearly internalized into the cell cytoplasm (see the lateral projections at 2 and 5 μM). These observations indicate that IBs expressing heterologous proteins bind and penetrate mammalian cells, keeping the conformational status and biological activity of the forming protein and maintaining their mechanical stability.

### Example 2

### Production and Characterization of IBs by the Aggregation of Hsp70

In view of the data obtained in EXAMPLE 1, the inventors investigated if a therapeutic protein carried by IBs could show biological effects in the cells exposed to IBs. To that end, the human Hsp70 chaperone, a potent inhibitor of cell apoptosis (Gamido et al. (2003) “HSP27 and HSP70: potentially oncogenic apoptosis inhibitors” Cell Cycle 2: 579-584), among other activities of therapeutic value (Calderwood et al. (2005) “Message in a bottle: role of the 70-kDa heat shock protein family in anti-tumor immunity” Eur. J. Immunol. 35: 2518-2527), was chosen as a model protein.

### Materials and Methods

2.1 Production of Inclusion Bodies—Following a protocol such as that described in section 1.1 of Example 1, duly adapted, inclusion bodies (IBs) were produced in strains of E. coli BL21 (DE3) transformed with a pReceiver-B01 commercial expression vector containing a N-His tag, a T7 promoter, and an ampicillin resistance gene (OmicsLink™ ORF Expression Ready Clone Catalog #EX-R0068-B1, GeneCopoeia, Rockville, Md.), expressing the human Hsp70 protein (Homo sapiens heat shock 70 kD protein 1B, HSPA1B, NCBI Reference Number: NM_{—}005346; Genbank GI: 167466172) with a Histidine-6 tag fused at the N-terminus. The bacterial cells were cultured in LB rich medium supplemented with 100 μg/ml of ampicillin, and the recombinant gene expression was induced by adding 1 mM IPTG. The IBs were clearly detectable 1 h after adding IPTG. The IBs formed by the aggregation of Hsp70 were purified following a procedure such as that described in section 1.2 of Example 1.

2.2 Apoptosis Assay—Cell apoptosis was determined by a flow cytometric assay with Annexin V-FITC57 by using an Annexin V-FITC Apoptosis Detection Kit (Roche). Exponential growth of the HL-60 cells (acute promielocytic leukemia, ATCC No. CLL-240) was adjusted to 3×105 cells/ml, seeded in six-well plates at 2.5 ml/well. Cells were exposed to the IC50 concentration of cisplatin (15.6 μM). Simultaneously to the cisplatin addition, 100 μl of different dilutions of inclusion bodies solutions were added to each well. As controls, the same amount of inclusion bodies was added to cells in the absence of cisplatin, for the purpose of detecting putative deleterious effect of IBs on cells. After 24 h of incubation, cells were subjected to staining with Annexin V-FITC and propidium iodide, as recommended by the manufacturer. The amount of apoptotic cells was determined by flow cytometry (FACSCalibur, BD Biosciences, San Jose, Calif.).

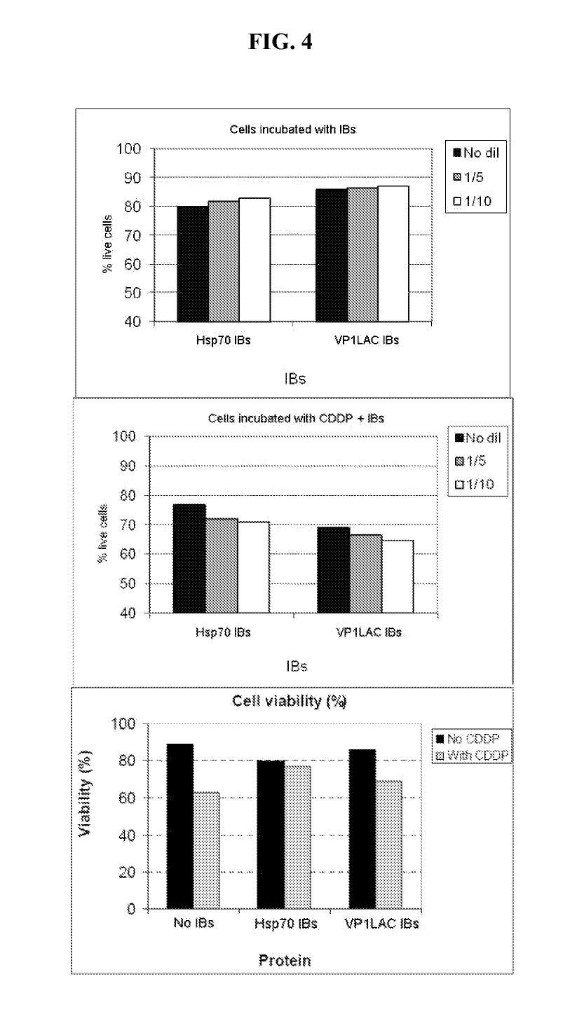

RESULTS—FIG. 4 shows, in the first place, the potential cytotoxic effects of Hsp70 IBs and also of VP1LAC (EXAMPLE 1) used as negative controls in the subsequent experiments, are first shown. As shown in FIG. 4A, cell growth was unaffected when cells were exposed to different types of IBs (FIG. 3). When cisplatin was added to the cultures, cell viability mediated by apoptosis was significantly reduced (FIG. 4B), and the same event was observed when cells were simultaneously exposed to VP1LAC IBs. However, in the presence of Hsp70 IBs, apoptotic events conducing to cell death were not evident, proving that the human Hsp70 chaperone contained in IBs was able to perform its natural biological activities. This cytoprotective effect was clearly dependent on the used concentration of IBs (FIG. 4C).

These data indicates that human cancer cells exposed to apoptosis-inducing chemotherapeutic agents such as cisplatin and treated subsequently with Hsp70 IBs maintain their viability. Furthermore, these observations show that proteins produced in insoluble IB form can be administered to cells, that these proteins are biologically active, and that the biological activity has a therapeutic effect. Also, they show that IBs are nanoparticles with therapeutic value, and which, in addition, are mechanically and functionally stable, fully biocompatible and easily producible in microbial cell factories.

### Example 3

### Dihydrofolate Reductase (DHFR) Inclusion Bodies (IB)

Dihydrofolate reductase converts dihydrofolate into tetrahydrofolate, a methyl group shuttle required for the de novo synthesis of purines, thymidylic acid, and certain amino acids. Deficiencies or alterations of DHFR have been linked to inherited spina bifida and neuronal tube defects, and to megaloblastic anemia. The average worldwide incidence of spina bifida is 1 case per 1000 births, but marked geographic variations occur. The highest rates are found in parts of the British Isles, mainly Ireland and Wales, where 3-4 cases of myelomeningocele per 1000 population have been reported, along with more than 6 cases of anencephaly (both live births and stillbirths) per 1000 population. The reported overall incidence of myelomeningocele in the British Isles is 2-3.5 cases per 1000 births. In France, Norway, Hungary, Czechoslovakia, Yugoslavia, and Japan, a low prevalence is reported: 0.1-0.6 cases per 1000 live births. In the United States, a declining prevalence has been noted, with the incidence being higher on the East Coast than on the West Coast. The prevalence in African Americans (0.1-0.4 case per 1000 live births) is lower than that in white Americans (1 case per 1000 live births). One would expect that treatment including the DHFR enzyme would allow recovering normal levels of folate, and therefore avoiding the appearance of the above mentioned pathologies, or recovery to normal function if clinical signs had already been detected before treatment with DHFR.

DHFR is also crucial in the regeneration of tetrahydrobiopterin (BH4), a cofactor of the nitric oxide synthase (eNOS) involved in the production of nitric oxide (NO). NO is a key element of the control of the vascular tone from the endothelium, and therefore of the arterial pressure. In experiments in mice, treatment with angiotensin II, a potent hypertensor) can be reverted by increasing the expression of DHFR. 15% of the worlwide population is diagnosed of having arterial hipertension. Treatment with DHFR could therefore allow to reduce arterial hypertension.

### Materials and Methods

3.1. Production of Inclusion Bodies: Culture samples of 20 ml are harvested by centrifugation at 5.000 g at 4° C. for 5 min, resuspended in lysis buffer (50 mM TrisHCl pH 8.1, 100 mM NaCl and 1 mM EDTA) and frozen at −80° C. After thawing, 100 μl, 100 mM of phenylmethanesulphonylfluoride (PMSF) (or other protease inhibitor) and 400 μl of 50 mg/mL lysozime are added and samples are incubated at 37° C. for 2 h. After the incubation, 100 μl of the same lysis buffer containing 0.5% Triton X-100 is added and incubated at room temperature for 1 h. Then, samples are disrupted using sonication or another disruption method, such as high pressure homogenization. After that, 5 μl of Nonidet P40 (NP-40) are added, and samples are incubated at 4° C. for 1 h. Then, DNA is removed with 15 μl of 1 mg/ml DNase and 15 μl 1M MgSO₄ for 45 min at 37° C. Finally, samples are centrifuged at 4° C. at 15000 g for 15 min, and the pellet containing pure IBs is washed once with 1 ml of lysis buffer containing 0.5% Triton X-100. After a final centrifugation at 15000 g for 15 min at 4° C., pellets are stored at −80° C. until analysis. All incubations are done under agitation. The volumes and incubation times used in this protocol are scaled up when using higher amounts of sample

3.2 Cell Culture: CHO (Chinese Hamster Ovary) DG44 cells (Invitrogen, Cat. No. 12609-012) are DHFR defective but can be grown in the presence of the complete medium DG44 (GIBCO), which contains purine precursors essential for DNA synthesis. At the beginning of the experiment, the former medium is substituted by OptiCHO (GIBCO), a medium without these precursors. Most CHO DG44 cells die after been cultured for a few days in OptiCHO medium.

3.3 Cell survival (MTT) assay: After the incubation of CHO DG44 cells for 72 h at 37° C. in OptiCHO or complete DG44 medium in a humidified 5% CO₂/95% air atmosphere, the number of cells is determined using the EZ4U kit (Biomedica, GmbH) following manufacturer instructions, and analyzed in the multilabel reader VICTOR³ V 1420 (Perkin Elmer). The reading absorbance is 450 nm, with 620 nm as reference, and the values obtained are standardized with respect to wells containing only medium. A pre-test to select time of incubation before saturation is carried out with the kit reagents; the optimal times being 2 h for 72 h cultures. All assays are done in triplicate. Data is expressed as the mean±SEM of the values from three experiments carried out per condition and evaluated statistically by a T-test analysis. The threshold level of significance chosen is p<0.05.

To evaluate the effect of DHFR IBs on cell viability, CHO DG44 cells are treated with DHFR IBs (1-1000 U/ml) just after OptiCHO replacement, and cell viability is assessed after 72 h of incubation. Treatment with soluble hDHFR (Sigma) is used as a positive control. The survival is expressed as a percentage to that obtained in CHO DG44 cells incubated for 72 h in complete medium.

RESULTS: After 72 h cultured in OptiCHO medium, CHO DG44 cell survival decreases by more than 50% relative to cells cultured in complete medium. The addition of DHFR IBs rescues cell death and improves cell survival with statistical significance, as seen in MTT assays.

### Example 4

### Catalase Inclusion Bodies

Catalase catalyzes the decomposition of hydrogen peroxide to water and oxygen. During the transformation of molecular oxygen to water, reactive oxygen species such as the superoxide radical, hydrogen peroxide and the hydroxyl radicals are generated. While only a weak oxidant, hydrogen peroxide has high potential to produce damage due to its ability to penetrate and spread freely across cell membranes. These species are capable of damaging DNA, protein and lipid membranes, and are known to be causative factors in degenerative diseases such as cancer. For defense against reactive oxygen species, cells contain antioxidative enzymes such as superoxide dismutase, catalase and several peroxidases, as well as antioxidants such as ascorbate, tocopherol and glutathione. Red blood cells contain large amounts of catalase and are believed to act as a sink for hydrogen peroxide and superoxide removal.

Catalase could therefore be used treat a variety of diseases where there is a need to reduce hydrogen peroxide, such as cancer, ulcerative colitis and Crohn's disease.

Catalase is also used in the food industry for removing hydrogen peroxide from milk prior to cheese production (“Catalase”. Worthington Enzyme Manual. Worthington Biochemical Corporation, Lakewood, N.J.). Another use is in food wrappers where it prevents food from oxidizing. Catalase is also used in the textile industry, removing hydrogen peroxide from fabrics to make sure the material is peroxide-free.

A minor use is in contact lens hygiene—a few lens-cleaning products disinfect the lens using a hydrogen peroxide solution; a solution containing catalase is then used to decompose the hydrogen peroxide before the lens is used again. Recently, catalase has also begun to be used in the aesthetics industry.

### Materials and Methods

4.1. Production of Inclusion Bodies: Culture samples of 20 ml are harvested by centrifugation at 5.000 g at 4° C. for 5 min, resuspended in lysis buffer (50 mM TrisHCl pH 8.1, 100 mM NaCl and 1 mM EDTA) and frozen at −80° C. After thawing, 100 μl, 100 mM of phenylmethanesulphonylfluoride (PMSF) (or other protease inhibitor) and 400 μl of 50 mg/mL lysozime are added and samples are incubated at 37° C. for 2 h. After the incubation, 100 μl of the same lysis buffer containing 0.5% Triton X-100 is added and incubated at room temperature for 1 h. Then, samples are disrupted using sonication or another disruption method, such as high pressure homogenization. After that, 5 μl of Nonidet P40 (NP-40) are added, and samples are incubated at 4° C. for 1 h. Then, DNA is removed with 15 μl of 1 mg/ml DNase and 15 μl 1M MgSO₄ for 45 min at 37° C. Finally, samples are centrifuged at 4° C. at 15000 g for 15 min, and the pellet containing pure IBs is washed once with 1 ml of lysis buffer containing 0.5% Triton X-100. After a final centrifugation at 15000 g for 15 min at 4° C., pellets are stored at −80° C. until analysis. All incubations are done under agitation. The volumes and incubation times used in this protocol are scaled up when using higher amounts of sample.

4.2 Cerebellar Granule Neuron Cultures: Cerebellar granule neuron (CGC) cultures are prepared as previously described (Valencia & Moran (2004) “Reactive oxygen species induce different cell death mechanisms in cultured neurons”. Free Radic. Biol. Med. 36:1112-25). Briefly, cell suspensions dissociated from 8-day-old rat cerebellum are plated in plastic dishes previously coated with poly-L-lysine (5 μg/ml). The culture medium contains basal Eagle's medium supplemented with 10% (v/v) heat-inactivated fetal calf serum, 2 mM glutamine, 25 mM KCl, 50 U/ml penicillin, and 50 μg/ml streptomycin. The culture dishes are incubated at 37° C. in a humidified 5% CO₂/95% air atmosphere. To avoid non-neuronal cells, cytosine arabinoside (10 μM) is added 20 h after seeding.

4.3 Induction of Hydrogen Peroxide: After 7-8 days in vitro, hydrogen peroxide formation (H₂O₂) is induced by incubating neurons with 6 mM glucose, and after 1 h glucose oxidase (GO 1-100 U/ml) is added.

4.4 Cell survival (MTT) Assay: After the incubation of neurons with glucose and glucose oxidase for six hours at 37° C. in a humidified 5% CO₂/95% air atmosphere, the number of cells is determined using the EZ4U kit (Biomedica, GmbH) following manufacturer instructions, and analyzed in the multilabel reader VICTOR³ V 1420 (Perkin Elmer). The reading absorbance is 450 nm, with 620 nm as reference, and the values obtained are standardized with respect to wells containing only medium. A pre-test to select time of incubation before saturation is carried out with the kit reagents. All assays are done in triplicate. Data is expressed as the mean±SEM of the values from the three experiments carried out per condition and evaluated statistically by a T-test analysis. The threshold level of significance is p<0.05.

To evaluate the effect of catalase IBs on cell viability, neurons are treated with glucose and glucose oxidase for six hours in the presence/absence of catalase inclusion bodies (1-1000 U/ml), and cell viability is assessed afterwards. Treatment with soluble catalase from bovine liver (Sigma) is used as a positive control. The survival is expressed as a percentage to that obtained in neurons without treatment.

RESULTS: Neuronal survival decreases by more than 50% after the induction of hydrogen peroxide formation (Valencia & Moran (2004) “Reactive oxygen species induce different cell death mechanisms in cultured neurons”. Free Radic. Biol. Med. 36:1112-1125), but if the neurons are incubated simultaneously with catalase IBs, their antioxidant activity has a protective effect and neuronal viability improves significantly as measured by the MTT assays.

### Example 5

### Interleukin-10 (IL-10) Inclusion Bodies

Interleukin-10 (IL-10 or IL10), also known as human cytokine synthesis inhibitory factor (CSIF), is an anti-inflammatory cytokine This cytokine is produced primarily by monocytes and to a lesser extent by lymphocytes. This cytokine has pleiotropic effects in immunoregulation and inflammation. It down-regulates the expression of Th1 cytokines, MHC class II antigens, and co-stimulatory molecules on macrophages. It also enhances B cell survival, proliferation, and antibody production. This cytokine can block NF-κB activity, and is involved in the regulation of the JAK-STAT signaling pathway. Knockout studies in mice suggested the function of this cytokine as an essential immunoregulator in the intestinal tract and indeed patients with Crohn's disease react favorably towards treatment with bacteria producing recombinant interleukin 10, showing the importance of interleukin 10 for counteracting excessive immunity in the human body (Braat et al. (2006) “A phase I trial with transgenic bacteria expressing interleukin-10 in Crohn's disease”. Clin. Gastroenterol. Hepatol. 4:754-9).

### Materials and Methods

5.1. Production of Inclusion Bodies: Culture samples of 20 ml are harvested by centrifugation at 5.000 g at 4° C. for 5 min, resuspended in lysis buffer (50 mM TrisHCl pH 8.1, 100 mM NaCl and 1 mM EDTA) and frozen at −80° C. After thawing, 100 μl, 100 mM of phenylmethanesulphonylfluoride (PMSF) (or other protease inhibitor) and 400 μl of 50 mg/mL lysozime are added and samples are incubated at 37° C. for 2 h. After the incubation, 100 μl of the same lysis buffer containing 0.5% Triton X-100 is added and incubated at room temperature for 1 h. Then, samples are disrupted using sonication or another disruption method, such as high pressure homogenization. After that, 5 μl of Nonidet P40 (NP-40) are added, and samples are incubated at 4° C. for 1 h. Then, DNA is removed with 15 μl of 1 mg/ml DNase and 15 μl 1M MgSO₄ for 45 min at 37° C. Finally, samples are centrifuged at 4° C. at 15000 g for 15 min, and the pellet containing pure IBs is washed once with 1 ml of lysis buffer containing 0.5% Triton X-100. After a final centrifugation at 15000 g for 15 min at 4° C., pellets are stored at −80° C. until analysis. All incubations are done under agitation. The volumes and incubation times used in this protocol are scaled up when using higher amounts of sample

5.2 MC/9 Cell Culture: The proliferation rate of IL-10-sensitive MC/9 (mouse mast) cells (ATTC: CRL-8306™) is used to estimate the agonistic activity of IL-10 IBs as described (Klompus et al. (2008) “A simple novel method for the preparation of noncovalent homodimeric, biologically active human interleukin 10 in Escherichia coli-enhancing protein expression by degenerate PCR of 5′ DNA in the open reading frame.” Protein Expr. Purif. 62:199-205) The cells are cultured at 37° C. in a 95%/5% air/CO₂ atmosphere in RPMI-1640 medium supplemented with 10% (w/v) FCS, 2 mM 1-glutamine, 2 mM nonessential amino acids, 50 IM β-mercaptoethanol, 30 ng/ml mouse IL-3 and 20 ng/ml mouse IL-4. Prior to the proliferation experiment, cells are washed in the same medium except that FCS is lowered to 3%, mouse IL-3 to 5 ng/ml and mouse IL-4 is omitted. The cells (15,000-20,000) are divided into 96-well plates (100 μ/well) and different amounts of hIL-10 IBs is added. The plates are then incubated for an additional 48 h, and the extent of proliferation is determined using the MTT method.

5.3 MC/9 Cell Proliferation Assay (MTT): After the incubation of MC/9 cells for 48 h at 37° C. in the resting medium mentioned above with or without IL-10 IBs in a humidified 5% CO₂/95% air atmosphere, the number of cells is determined using the EZ4U kit (Biomedica, GmbH) following manufacturer instructions, and analyzed in the multilabel reader VICTOR³ V 1420 (Perkin Elmer). The reading absorbance is 450 nm, with 620 nm as reference, and the values obtained are standardized with respect to wells containing only medium. A pre-test to select time of incubation before saturation is carried out with the kit reagents. All assays are done in triplicate. Treatment with soluble hIL-10 (Sigma) is used as a positive control. The survival is expressed as a percentage to that obtained in cells in complete medium. Cells cultured for 48 h in resting medium are used as negative control. Data is expressed as the mean±SEM of the values from the three experiments carried out per condition and evaluated statistically by a T-test analysis. The threshold level of significance is p<0.05.

RESULTS: IL-10 IBs have biological activity by increasing significantly proliferation of MC/9 cells when added to the resting medium, as seen in MTT assays.

### Example 6

### Therapeutic Efficacy vs. Murine Colitis

Inflammatory bowel disease (IBD) is a group of inflammatory conditions of the colon and small intestine. The major types of IBD are Crohn's disease and ulcerative colitis. The main forms of IBD are Crohn's disease and ulcerative colitis (UC). Accounting for far fewer cases are other forms of IBD: Collagenous colitis, lymphocytic colitis, ischaemic colitis, diversion colitis, Behçet's syndrome, or indeterminate colitis. Depending on the level of severity, IBD may require immunosuppression to control the symptom, such as prednisone, TNF inhibition, azathioprine (Imuran), methotrexate, or 6-mercaptopurine. More commonly, treatment of IBD requires a form of mesalamine. Often, steroids are used to control disease flares and were once acceptable as a maintenance drug. In use for several years in Crohn's disease patients and recently in patients with ulcerative colitis, biologicals have been used such as TNF inhibitors. Severe cases may require surgery, such as bowel resection, strictureplasty or a temporary or permanent colostomy or ileostomy.

Prebiotics and probiotics are showing increasing promise as treatments for IBD (Furrie et al. (2005). “Synbiotic therapy (Bifidobacterium longum/Synergy 1) initiates resolution of inflammation in patients with active ulcerative colitis: a randomised controlled pilot trial” Gut 54:242-9) and in some studies have proven to be as effective as prescription drugs (Kruis et al. (2004). “Maintaining remission of ulcerative colitis with the probiotic Escherichia coli Nissle 1917 is as effective as with standard mesalazine”. Gut 53:1617-23).

### Materials and Methods

6.1. Production of Inclusion Bodies: Culture samples of 20 ml are harvested by centrifugation at 5.000 g at 4° C. for 5 min, resuspended in lysis buffer (50 mM TrisHCl pH 8.1, 100 mM NaCl and 1 mM EDTA) and frozen at −80° C. After thawing, 100 μl, 100 mM of phenylmethanesulphonylfluoride (PMSF) (or other protease inhibitor) and 400 μl of 50 mg/mL lysozime are added and samples are incubated at 37° C. for 2 h. After the incubation, 100 μl of the same lysis buffer containing 0.5% Triton X-100 is added and incubated at room temperature for 1 h. Then, samples are disrupted using sonication or another disruption method, such as high pressure homogenization. After that, 5 μl of Nonidet P40 (NP-40) are added, and samples are incubated at 4° C. for 1 h. Then, DNA is removed with 15 μl of 1 mg/ml DNase and 15 μl 1M MgSO₄ for 45 min at 37° C. Finally, samples are centrifuged at 4° C. at 15000 g for 15 min, and the pellet containing pure IBs is washed once with 1 ml of lysis buffer containing 0.5% Triton X-100. After a final centrifugation at 15000 g for 15 min at 4° C., pellets are stored at −80° C. until analysis. All incubations are done under agitation. The volumes and incubation times used in this protocol are scaled up when using higher amounts of sample

6.2 Animal model: To induce colitis, C57B1/6 mice receive dextran sulphate sodium (DSS) in their drinking water for five consecutive days. This DSS regime causes a mild-severe with weight loss, rectal mucus, diarrhea, bloody feces and changes in animal's aspect and behavior, but no mortality within the colitic control group. C57B1/6 mice subjected to a DSS regime are a well established animal models of human inflammatory bowel diseases such as Crohn's disease and ulcerative colitis (see, e.g., Pizarro et al. (2003) “Mouse models for the study of Crohn's disease” Trends in Molecular Medicine 9:218-22; Yan et al. (2009) “Temporal and Spatial Analysis of Clinical and Molecular Parameters in Dextran Sodium Sulfate Induced Colitis”. PLoS ONE 4: e6073; Melgar et al. (2008) “Validation of murine dextran sulfate sodium-induced colitis using four therapeutic agents for human inflammatory bowel disease” International Immunopharmacology 8:836-44; Axelsson et al. (1998) “Experimental colitis induced by dextransulphate sodium in mice: beneficial effects of sulphasalazine and olsalazine” Aliment Pharmacol. Ther. 12:925-34; Baker (2005) “Safety of balsalazide therapy in the treatment of inflammatory bowel disease” Rev. Gastroenterol. Disord. 5:135-41; Oz et al. (2005) “Antioxidants as novel therapy in a murine model of colitis” J. Nutr. Biochem. 16: 297-304).

Treatments A or B, wherein A and B involve the administration of IB-containing compositions capable of treating or ameliorating the symptoms associated with ulcerative colitis (e.g., purified IBs or cells containing IBs), are given daily (gavage) for 12 consecutive days. DSS is given from day 5 to day 10. The animals are killed 2 days after DSS cessation. There are at least 10 animals per group (see TABLE 1).

TABLE 1

| | | | Blood/ | Frozen | Fixed |
|---|---|---|---|---|---|
| Group | Day 0-12 | Day 5-10 | serum | tissue | tissue |
| Healthy | Daily gavage | Plain | SAA | MPO | Histopa- |
| controls | vehicle | drinking | Cytokines | Cytokines | thology |
| | | water | | | |
| Colitic | Daily gavage | DSS in | SAA | MPO | Histopa- |
| controls | vehicle | drinking | Cytokines | Cytokines | thology |
| | | water | | | |
| treatment | Daily gavage | DSS in | SAA | MPO | Histopa- |
| A+ colitis | treatment A | drinking | Cytokines | Cytokines | thology |
| | | water | | | |
| Treatment | Daily gavage | DSS in | SAA | MPO | Histopa- |
| B+ colitis | treatment B | drinking | Cytokines | Cytokines | thology |
| | | water | | | |

6.3 Variables Measured: Throughout the study, animals are examined daily to evaluate water/DSS and food consumption. An individual Disease Activity Index (DAI) is assigned daily (DAI includes scores of (i) weight loss (0-4 points), (ii) stool score (0-4 points), (iii) animals aspect (0-3 points) and (iv) blood in feces (0-3 points).

At endpoint, blood is extracted by heart puncture. Plasma samples are kept frozen until assay. SAA (Serum amyloid A protein), GSH (glutathione), and cytokine level assays are performed using the plasma samples.

6.4 Tissue collection and inspection: Stomach and small intestine, colon and associated lymph nodes, and spleen. Macroscopic inspection of aforementioned organs is performed. The colon is opened longitudinally and cut in two pieces. One is rolled and fixed in paraformaldehide solution and the other is snap frozen in liquid nitrogen and stored at −80° C. until assay. Half of the spleen is fixed in PFA and half is snap frozen in liquid nitrogen and stored at −80° C. until assay.

6.5 Assays performed using protein extracts of colonic tissue (ELISA): Tissue extracts are examined for levels of myeloperoxidase activity, as well as for levels of cytokines such as IL1β, IL6, IL12, and TNF-α.

6.6 Histopathology: Section of collected tissue are fixed with paraformaldehyde (PFA), embedded in paraffin, and Haematoxylin/Eosin (H/E) stained.

RESULTS: Visual tissue inspection, immunoassays performed on protein extracts, and histopathological analysis of samples collected from IB-treated animals and from controls show that samples from IB-treated animals are significantly more similar to healthy controls than to colitic controls. Thus, IB therapy treats or ameliorates the symptoms associated with ulcerative colitis

All publications such a textbooks, journal articles, Genbank or other sequence database entries, published applications and patent applications mentioned in this specification are herein incorporated by reference to the same extent as if each individual publication or patent was specifically and individually indicated to be incorporated by reference.

What is claimed is:

1. A method to deliver a therapeutic protein to a subject in need thereof comprising administering a non-solubilized inclusion body wherein said inclusion body comprises said therapeutic protein, and wherein said therapeutic protein is not a vaccine immunogen.

2. The method of claim 1, wherein said inclusion body is insoluble.

3. The method of claim 1, wherein said inclusion body is internalized by a target cell.

4. The method of claim 1, wherein said inclusion body is administered orally.

5. The method of claim 1, wherein said therapeutic protein is biologically active.

6. The method of claim 1, wherein said therapeutic protein is a recombinant protein.

7. The method of claim 1, wherein said therapeutic protein is selected from the group consisting of erythropoietin (EPO), corticotropin-releasing hormone (CRH), growth hormone-releasing hormone (GHRH), gonadotropin-releasing hormone (GnRH), thyrotropin-releasing hormone (TRH), prolactin-releasing hormone (PRH), melanotropin-releasing hormone (MRH), prolactin-inhibiting hormone (PIH), somatostatin, adrenocorticotropic hormone (ACTH), somatotropin or growth hormone (GH), luteinizing hormone (LH), follicle-stimulating hormone (FSH), thyrotropin (TSH or thyroid-stimulating hormone), prolactin, oxytocin, antidiuretic hormone (ADH or vasopressin), melatonin, Müllerian inhibiting factor, calcitonin, parathyroid hormone, gastrin, cholecystokinin (CCK), secretin, insulin-like growth factor type I (IGF-I), insulin-like growth factor type II (IGF-II), atrial natriuretic peptide (ANP), human chorionic gonadotropin (hCG), insulin, glucagon, somatostatin, pancreatic polypeptide (PP), leptin, neuropeptide Y, renin, angiotensin I, angiotensin II, factor VIII, factor IX, tissue factor, factor VII, factor X, thrombin, factor V, factor XI, factor XIII, interleukin 1 (IL-1), Tumor Necrosis Factor Alpha (TNF-α), interleukin 6 (IL-6), interleukin 8 (IL-8 and chemokines), interleukin 12 (IL-12), interleukin 16 (IL-16), interferons alpha, beta, gamma, nerve growth factor (NGF), platelet-derived growth factor (PDGF), transforming growth factor beta (TGF-beta), bone morphogenetic proteins (BMPs), fibroblast growth factors (FGF and KGF), epidermal growth factor (EGF and those related thereto), vascular endothelial growth factor (VEGF), granulocyte colony-stimulating factor (G-CSF), glial growth factor, keratinocyte growth factor, endothelial growth factor, alpha-1 antitrypsin, tumor necrosis factor, granulocyte-macrophage colony-stimulating factor (GM-CSF), cyclosporine, fibrinogen, lactoferrin, tissue-type plasminogen activator (tPA), chymotrypsin, immunoglobins, hirudin, superoxide dismutase, imiglucerase, and chaperones.

8. The method of claim 6, wherein said recombinant protein is expressed in cells selected from the group consisting of bacteria, yeasts, insect cells, and mammalian cells.

9. The method of claim 6, wherein said recombinant protein is a heterologous protein.

10. The method of claim 6, wherein said recombinant protein is a homologous protein.

11. The method of claim 6, wherein said recombinant protein is conjugated to an inclusion body-inducing polypeptide.

12. The method of claim 11, wherein said inclusion body-inducing polypeptide comprises the VP1 pentamer-forming capsid protein of Foot and Mouth Disease Virus (FMDV) or a fragment therein.

13. The method of claim 6, wherein said recombinant protein is conjugated to a protein tag.

14. The method of claim 13, wherein said protein tag is a His6-tag.

15. The method of claim 1, wherein the recombinant protein is an antiapoptotic protein for the treatment of diseases associated with an increase in apoptosis.

16. The method of claim 15, wherein said antiapoptotic protein is a chaperone.

17. The method of claim 16, wherein said chaperone is Hsp70.

18. The method of claim 17, wherein the disease associated with an increase in apoptosis is selected from the group consisting of cancer, AIDS, Alzheimer's disease, Parkinson's disease, amyotrophic lateral sclerosis, retinitis pigmentosa, cerebellar degeneration, myelodysplastic syndromes, aplastic anemia, ischemic injury, myocardial infarction, apoplexy, reperfusion injury, and liver damage.

19. The method of claim 17, wherein the disease is human leukemia or cervical cancer.

20. The method of claim 15, wherein said apoptosis is caused by chemotherapy with an anticancer agent.

21. The method of claim 20, wherein said anticancer agent is cisplatin.

22. A cell comprising the therapeutic protein delivered according to the method of claim 1.

23. The cell of claim 22, wherein said cell is an autologous cell.

24. A method for treatment of a disease or condition that benefits from treatment with therapeutic proteins in a patient, the method comprising administering to said patient an effective amount of the therapeutic protein according to the method of claim 1.

25. A method for treatment of a disease or condition that benefits from treatment with therapeutic proteins in a patient, the method comprising administering to said patient an effective amount of the cell according to claim 23.

26. A method for treatment of a disease or condition that benefits from treatment with therapeutic proteins in a patient, the method comprising administering to said patient an effective amount of a cell, wherein said cell expresses a therapeutic protein in inclusion body form, and wherein said therapeutic protein is not a vaccine immunogen.

27. A method for treatment of a disease or condition that benefits from treatment with therapeutic proteins in a patient, the method comprising the steps of:
(a) extracting cells from said patient;
(b) contacting said cells with the therapeutic protein according to the method of claim 1; and,
(c) implanting said cells containing said therapeutic protein into the patient.

28. A pharmaceutical formulation comprising a therapeutic protein in inclusion body form and a pharmacologically acceptable excipient.

29. A pharmaceutical formulation comprising a pharmacologically acceptable excipient and a cell, wherein said cell contains a therapeutic protein in inclusion body form.

30. A pharmaceutical formulation comprising a pharmacologically acceptable excipient and a cell, wherein said cell comprises a polynucleotide sequence encoding a therapeutic protein wherein said therapeutic protein forms inclusion bodies upon expression.
